(19) United States
(12) Patent Application Publication
(10) Pub. No.: US 2013/0231583 A1
(43) Pub. Date: Sep. 5, 2013

(54) Methods and Compositions for Injection Delivery

(71) Applicant: Donna Rekkerth, Scottsville, NY (US)

(72) Inventor: Donna Rekkerth, Scottsville, NY (US)

(21) Appl. No.: 13/780,972

(22) Filed: Feb. 28, 2013

Related U.S. Application Data
(60) Provisional application No. 61/606,299, filed on Mar. 2, 2012.

(51) Int. Cl. A61B 5/00; A61M 5/42

(52) U.S. Cl. CPC A61B 5/445; A61M 5/427
USPC 600/556; 604/506

(57) ABSTRACT

The present invention discloses methods, devices and systems for intradermal delivery comprising injection delivery guide devices as described herein. An injection delivery guide device may comprise a carrier member and at least one indentation applicator. Such methods, devices and systems are useful in intradermal administration of solutions comprising active agents.

### RELATED APPLICATIONS

The present application claims the priority of and the benefit of the filing date of U.S. Provisional Patent Application Ser. No. 61/606,299, filed Mar. 2, 2012, which is herein incorporated in its entirety.

### TECHNICAL FIELD

The present invention relates to methods and devices for injection delivery, particularly devices that provide a guide for injection, such as intradermal delivery.

### BACKGROUND OF THE INVENTION

Intradermal delivery of therapeutic or other solutions is a common medical procedure. There are difficulties in applying a known amount of the solution to the intradermal space reliably, and this is especially a problem in skin testing. Intradermal skin testing is a unique method of allergy testing that provides a quantitative, as well as qualitative measurement of immediate hypersensitivity. Generally, a series of allergens are injected into the skin, preferably intradermally. The person or animal who was injected will respond with an acute hypersensitivity response to the allergens to which the person or animal is allergic. Solutions of allergens to which the person or animal is not allergic will not show a hypersensitivity response.

Accurate interpretation of the intradermal test results depends on the proficiency of the person performing the intradermal test. The proficiency relies on the ability of the person to intradermally inject a sufficient volume of a potentially allergenic solution in a site in the skin to create a uniform wheal with a sharp demarcated edge. If the initial intradermal wheal formed by the injected solution is not of a uniform size, the interpretation of the skin test response measurement may be inaccurate, leading to a misdiagnosis of the immune condition of the person receiving the solutions.

What is needed are methods and devices for uniform injections, for example, that aid in performing intradermal injections and provide for uniform administration of solutions by an intradermal route of administration.

### SUMMARY

The present invention is directed to methods and devices for uniform injection, for example, intradermal injection. A device of the present invention comprises an injection delivery guide comprising at least one indentation applicator that, when applied to the skin of a human or animal, makes a defined indentation in the skin. The indentation serves as a template for injection of solution into the intradermal space of the indentation so that a constant amount of solution is applied in a uniform shape.

A method of the present invention comprises providing a device as disclosed herein to a surface, applying a sufficient force to the device for a sufficient time period to create in the surface one or more indentations. Indentations made in the surface may be used as a template for guiding the location and amount of a solution to be injected into an area defined by or created by the indentation. An example of a method of the present invention comprises providing a devices as disclosed herein to create indentations in skin, so that the indentation forms a design of indented skin that defines an area for injection, a template for injections that are uniform in size and amount. For example, an allergenic solution may be injected in the defined area.

### DESCRIPTION OF THE DRAWINGS

FIG. 1A-E shows an exemplary embodiment of an injection guide, for example for intradermal delivery, of the present invention.

FIG. 2A-E shows an exemplary embodiment of an injection guide, for example for intradermal delivery, of the present invention.

FIG. 3A-B show exemplary embodiments of an injection guide of the present invention, wherein the carrier member is a wheel.

FIG. 4A-B show exemplary embodiments of an injection guide of the present invention, wherein the carrier member is curved.

### DETAILED DESCRIPTION

The present invention is directed to methods and devices for an injection delivery guide. Devices of the present invention comprise at least one indentation applicator. An indentation applicator comprises at least one end for contacting the surface of a body, for example, for contacting the skin of a human or animal. The surface or skin contacting end of an indentation applicator is not capable of cutting or puncturing the surface contacted, and is generally smooth and blunt. When a sufficient pressure is applied to the applicator so that a surface is contacted, for a sufficient time period, the contacting end of the applicator leaves the impression of the contacting end as an indentation in the surface, such as skin, but does not cut or puncture the skin. As used herein, a sufficient time period can be determined by experimentation that is easily performed so that an indentation remains in the surface long enough to be visible for the period of time that injections are performed. A sufficient pressure may be by experimentation that is easily performed so that an indentation remains in the surface long enough to be visible for the period time that injections are performed. It is contemplated that anyone skilled in healthcare procedures can determine a sufficient time and a sufficient pressure after a few times of using the device and adapt such time and pressure for individuals or particular surfaces to be treated.

Methods of the present invention comprise providing an indentation in a surface, such as the skin of a human or animal, for applying a solution at the indentation, such as by intradermal administration. For example, a method of guided injection comprises providing to a surface, with sufficient force, to, for example, skin of a human or animal, a device of the present invention comprising at least one indentation applicator having a first end for contacting a surface and a second end affixed to a carrier member, wherein at least one indentation applicator first end contacts the surface and forms an indentation in the surface; removing the at least one indentation applicator from the surface to reveal one or more indentations in the surface. A method of injection into the surface may further comprise, a) inserting a needle of a syringe containing a liquid at the exterior edge of the indentation into the surface, for example where the suface is skin, into a desired depth into the skin, such as the superficial intradermal layer of the skin; and b) injecting the liquid, for example, into the intradermal layer, such that the liquid fills at least a portion of an area defined by the indentation.

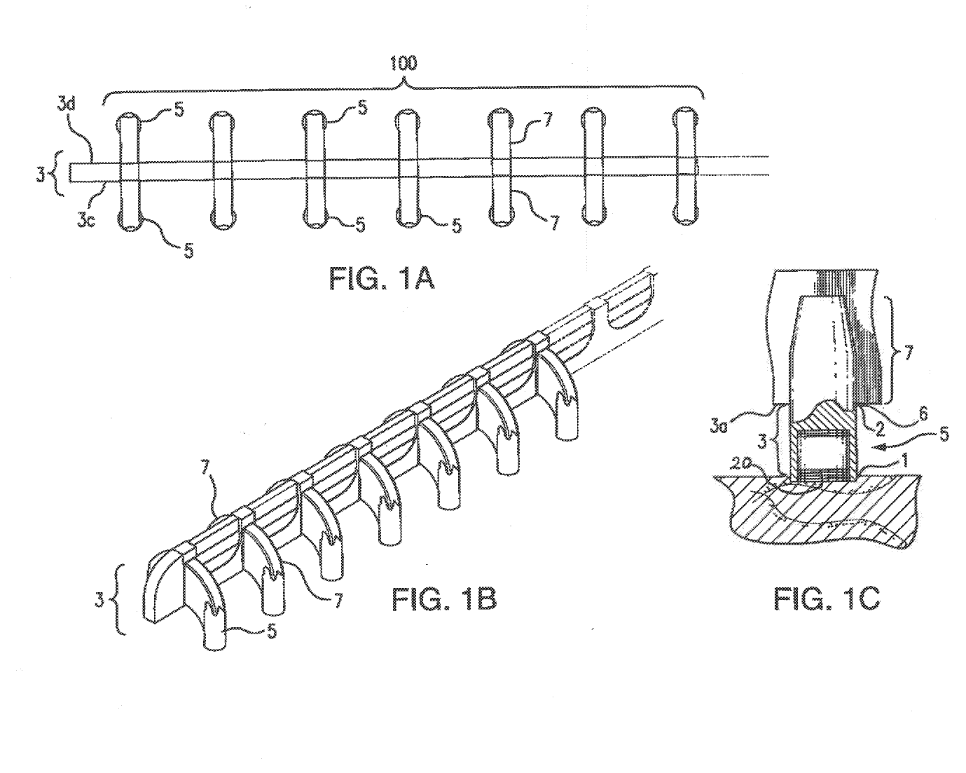

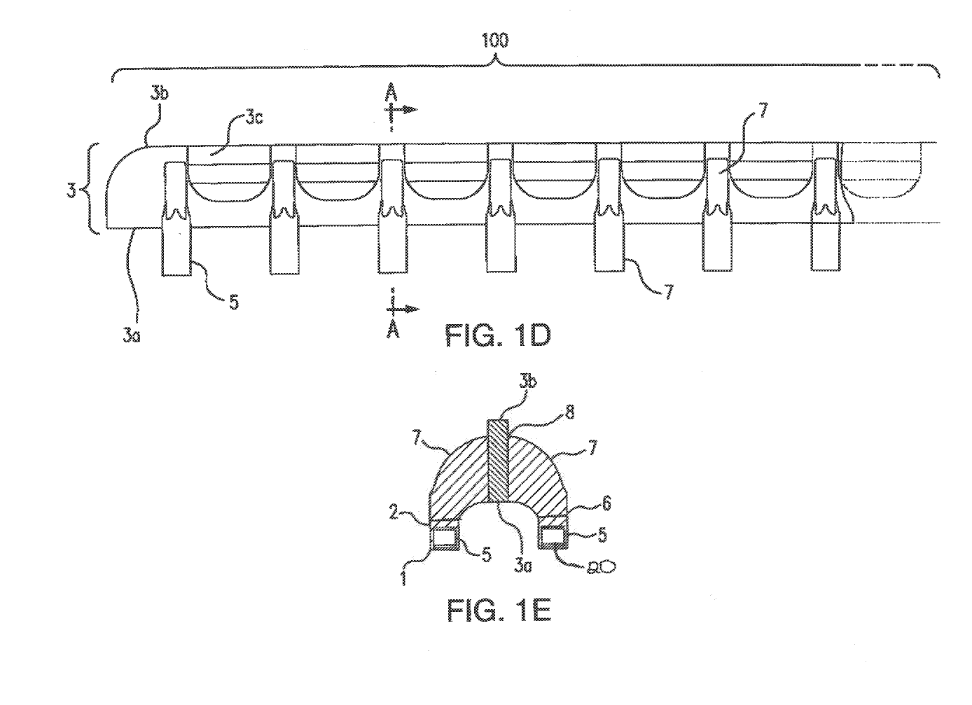

An exemplary embodiment of an injection delivery guide is shown in FIG. 1A-E. In FIG. 1A, a top view, injection delivery guide 100 is shown having a plurality of indentation applicators 5 affixed to carrier member 3. As shown, connecting element 7 connects indentation applicator 5 and carrier member 3. FIG. 1B shows a perspective view of injection delivery guide 100, having a plurality of indentation applicators 5 affixed to carrier member 3, wherein connecting element 7 connects indentation applicator 5 and carrier member 3.

FIG. 1C shows indentation applicator 5 having a first end 1 that contacts a surface, such as skin, and a second end 2 that is affixed to a first end 6 of connecting element 7 having a second end 8 which is affixed to carrier member 3. Optionally, in embodiments of the present invention, one or more of an indentation applicator 5 and/or a connecting element 7 may be removably affixed to each other and/or to carrier member 3. In an aspect, indentation applicator 5 may or may not be removably affixed to carrier member 3, or may or may not be removably affixed to connecting element 7. In an aspect, connecting element 7 may or may not be removably affixed to carrier member 3 or may or may not be removably affixed to indentation applicator 5. In an aspect, at least one of a set of indentation applicator 5 and connecting element 7 may or may not be removably affixed to each other and/or to carrier member 3. Carrier member 3 may have one or more receiver sites 9 where removably affixable indention applicators can be removably affixed, or where connecting elements 7 may be removably affixed, or a connecting element 7 may be removably affixed to an indentation applicator 5. As used herein, a receiving site provides one portion of an affixing unit, and the term is not to be seen as restricting the structure of the site, as in not just a receiving-type structure. For example, an affixing unit may comprise a male and a female luer lock portions, wherein the female luer lock is one portion of one component and the male leur lock is another portion of another component, and when mated together, removably affix one component to another component, such as a connecting element 7 at first end 6 to second end 2 of an indentation applicator 5. Generally an affixing unit may be made of two portions, each of which is a part of one component, but affixing units made of multiple pieces or more than two portions, which are used to removably affix two components of a device, are contemplated by the present invention. Additionally, extension members (not shown) may be present on one or both ends of connecting element 7 or on one or both ends of indentation applicator 5, and may be used to lengthen a connecting element or an indentation applicator, or to provide a directional orientation for one or both of the connecting element and the indentation applicator. Ends of a removable indentation applicator 5, such as second end 2, or a removable connecting element 7, such as first end 6 and second end 8 may function as receiving sites and provide a portion of an affixing unit.

As shown in FIG. 1C, the body 13 of indentation applicator 5 is at least partially hollow such that the interior of first end 1 is an open space 20 defined by the wall of indentation applicator 5. When first end 1 contacts the skin or other surface with force sufficient to cause an indentation in the skin or other surface, the indentation formed in the skin comprises a depressed area in the shape of a circle, or an annulus or ring-shaped, which is the shape of the first end 1 shown in FIG. 1C. Alternatively, first end 1 may be elliptically shaped, or any other desired shape. The indentation shape made by first end 1 in a surface creates the template or guide for injection of a fluid into the surface.

After contact with a first end 1, the surface, such as skin, comprises a depressed area in the shape of first end 1, such as with an indentation applicator of FIG. 1, the depressed area is shaped like an annulus or ring, which contains in its interior a semi-sphere of skin, where the skin was not depressed, and surrounding the depressed annulus, the skin is either at its normal height or slightly raised near the annulus due to response to formation of the indentation. The skin within the depressed ring or annulus may be slightly raised or may be at its normal height. As used herein, an indentation is the depressed area of the surface, such as skin, that was formed where the surface was contacted by first end 1 of an indentation applicator with force sufficient to form a depression in the surface and for a time sufficient to cause the surface to respond by forming an indentation. An indentation may comprise a annular or ring shape as described herein by the application of an indentation applicator that is a hollow cylinder at its first end 1, or an indentation may be formed by an indentation applicator having any desired shape. For example, an indentation applicator may be solid, such that first end 1 is shaped as a solid disk when viewed directly at the end, and the indentation formed is a depressed disk, such that the entire area within the circular border is depressed and the skin or surface not contacted, outside the circular disk, is either slightly raised or at its normal skin or surface height.

The body 13 of an indentation applicator 5 of the present invention may have any desired shape that aids in transmission of the force applied to the handle or the carrier member to the first end of one or more indentation applicators. As shown in FIG. 1, the body is shaped as a cylinder. The shape may be any known shape. For example, the present invention contemplates indentation applicators wherein at least one indentation applicator has a frustoconical shaped body portion, wherein one end is the apex end and the second end is the base end and the applicator further comprises a cylindrical extension extending from the apex end wherein one end of the extension contacts a surface and a cylindrical extension extending from the base end for affixing to the carrier member. An aspect of the invention comprises an indentation applicator having a frustoconical shaped body portion wherein the first end (for contacting the surface) is the apex end and the second end is the base end; and wherein the apex end is for contacting the skin and a cylindrical extension connecting from the base end for affixing to the carrier member An aspect of the invention comprises an indentation applicator having a frustoconical shaped body, wherein the first end is the apex end and the second end is the base end; wherein the apex end is for contacting the skin and the base end is for affixing to the carrier member. Extensions may or may not be removably affixed to its adjacent component or to a carrier.

As shown in FIG. 1D, a side view of a device of the present invention, a plurality of indentation applicators 5 are affixed to carrier member 3 via a connecting element 7 that is placed between an individual indentation applicator 5 and carrier member 3. Carrier member 3 may have one or more sides, for example, as shown in FIG. 1D, a side that faces the surface to be contacted, a surface facing side 3a, and a side opposite that faces away from the surface to be contacted, opposing surface side 3b. Carrier member 3 may have an other side 3c, and its complement side (not shown) 3d, depending on the shape of the carrier member, a carrier may have sides that are perpendicular to a surface facing side 3a and/or a opposing surface side 3b. A handle may be affixed to side 3b. In an embodiment, as shown in FIG. 1D, connecting element 7 and/or indentation applicator 5 may be affixed on both other side 3c and its complement side 3d in a paired fashion. Alternatively, the pairs may be staggered along the longitudinal axis of carrier member 3.

FIG. 1E shows a cross-section view at A-A of 1D, wherein two indentation applicators 5 are shown, each affixed to a connecting element 7 having two ends, a first end 6 affixed to second end 2 of indentation applicator 5, and a second end 8 affixed to carrier member 3. As shown, connecting element 7 has an arcuate shape, but this is exemplary, and a connecting element, if present may have any shape desired that will allow for connecting an indentation applicator 5 to carrier member 3. For example, a connecting element 7 may be a squared right angle connecting member.

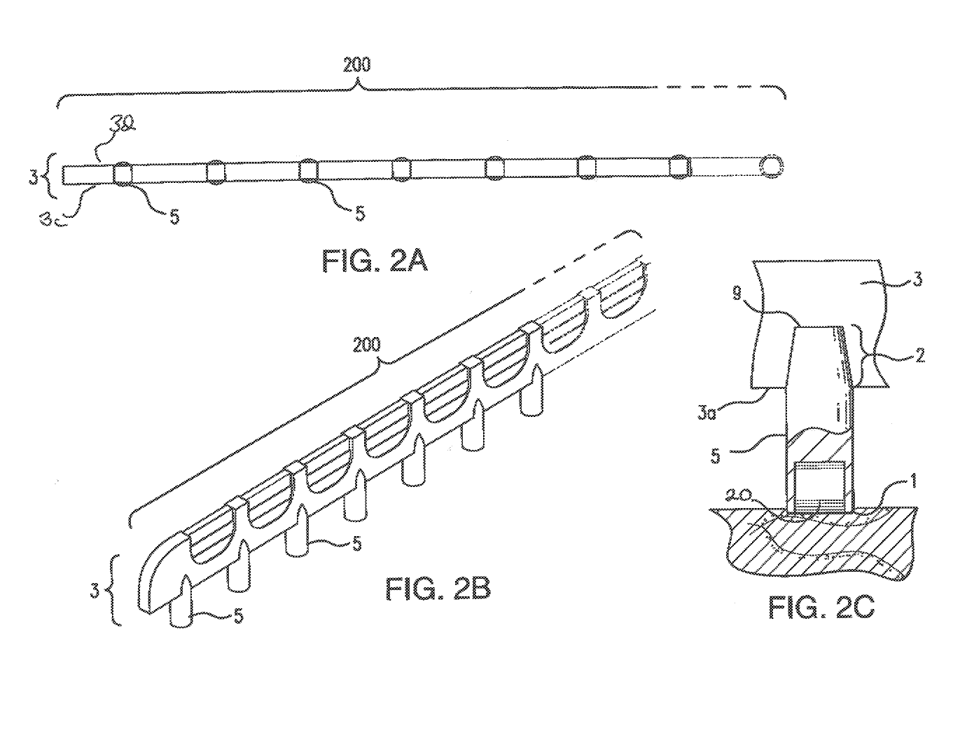

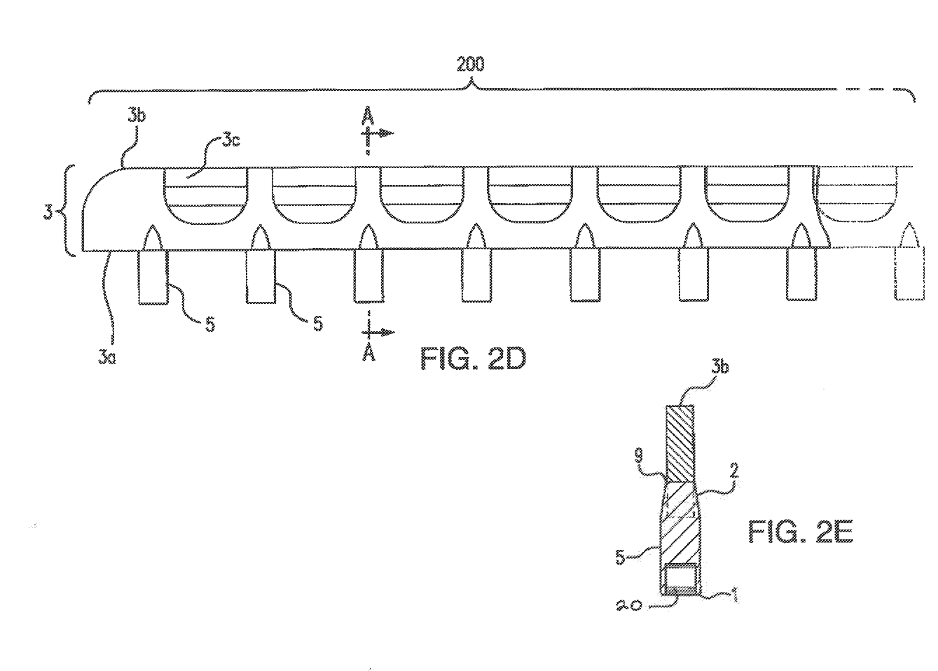

An exemplary embodiment of an injection delivery guide is shown in FIG. 2A-E. In FIG. 2A, a top view, injection delivery guide 200 is shown having a plurality of indentation applicators 5 affixed to carrier member 3. FIG. 2B shows a perspective view of injection delivery guide 200, having a plurality of indentation applicators 5 affixed to carrier member 3.

FIG. 2C shows indentation applicators 5 having a first end 1 that contacts a surface, such as skin, and a second end 2 that contacts carrier member 3. Optionally, indentation applicators 5 may be removably affixed to carrier member 3. Carrier member 3 may have one or more receiver sites 9 where removable applicators can be removably affixed.

As shown in FIG. 2C, the indentation applicator 5 is at least partially hollow such that the interior of first end 1 is an open space 20 defined by the wall of indentation applicator 5. When first end 1 contacts the skin or other surface with force sufficient to cause an indentation in the skin or other surface, the indentation formed in the skin comprises a depressed area in the shape of a circle, or an annulus or is ring-shaped, which is the shape of the first end 1 shown in FIG. 2C. For example, when the surface contacted is skin, and the depressed annulus describes in its interior a semi-sphere of skin, where the skin was not depressed, and surrounding the depressed annulus (indentation), the skin is either at its normal height or slightly raised near the circle due to response to formation of the indentation. The skin within the depressed circle may be slightly raised or may be at its normal height. Alternatively, first end 1 may be elliptically shaped, or any other desired shape.

As disclosed, the body 13 of an indentation applicator of the present invention may have any desired shape that aids in transmission of the force applied to the handle or the carrier member to the first end of one or more indentation applicators. As shown in FIG. 2, the body is shaped as a cylinder. The shape of an indentation applicator 5 may be any known shape.

As shown in FIG. 2D, a side view, a plurality of indentation applicators 5 is affixed to carrier member 3. Carrier member 3 may have one or more sides, for example, as shown in FIG. 2D, a side that faces the surface to be contacted, a surface facing side 3a, and a side opposite that faces away from the surface to be contacted, opposing surface side 3b. Carrier member 3 may have an other side 3c, and its complement side (not shown) 3d, depending on the shape of the carrier member, such as sides that are perpendicular to a surface facing side 3a and/or a opposing surface side 3b. A handle (not shown) may be affixed to side 3b. In an embodiment, as shown in FIG. 2D, indentation applicator 5 may be affixed to carrier member 3 directly, and extend perpendicularly from surface side 3a at regular intervals, or in a different or no particular pattern.

FIG. 2E shows a cross-section view at A-A of 1D, wherein one indentation applicators 5 is shown, having second end 2 of indentation applicator 5 affixed to carrier member 3, and first end 1 for contacting a surface. Indentation applicator 5 is affixed to carrier member 3 at receiving site 9 of carrier member 3.

An aspect of the present invention comprises a device comprising one or more indentation applicator 5 that is removably attached to carrier member 3 or to connecting element 7. A removable indentation applicator 5 of the present invention may snap fit or friction fit into a connecting element or a carrier member, or may be affixed by clips, clamps, having a screw end that mates with a screw receiver in the connecting element or a carrier member, having a female or male connection that mates with the opposite receiving connection in the connecting element or a carrier member, or other known ways of removably affixing one element of a device to another element of a device. In a similar manner, a connecting element may be removably attached to one or both of the indentation applicator 5 and/or the carrier member 3, and may be removably attached by any removable affixation elements, such as those listed above and others.

A carrier member may further comprise a handle for gripping the carrier member and for ease in applying force to the first end 1 of an indentation applicator 5. The handle may be of the same or different material as the carrier member, and may be molded with the carrier member to form one piece. Alternatively, a handle may be removably attached to a carrier member, and such ways for removably attaching a handle are known to those skilled in the art.

As shown in FIG. 1, carrier member 3 is a solid planar bar with indentation applicators arranged in parallel pairs, with one on each side of the bar. This is an exemplary arrangement of the indentation applicators, and a device of the present invention may comprise from 1 to about 30 indentation applicators in any desired arrangement. For example, an injection indentation guide of the present invention may comprise from 1 to 10 indentation applicators, from about 1 to 15 indentation applicators, from about 1 to 20 indentation applicators, from about 5 to 20 indentation applicators, from about 20 to 30 indentation applicators, or may comprise 1 to 5 indentation applicators, or may comprise 1 indentation applicator, 2 indentation applicators, 3 indentation applicators, 4 indentation applicators, 5 indentation applicators, 10 indentation applicators, 15 indentation applicators, 20 indentation applicators, 50 indentation applicators, and all numbers there inbetween 1 and 100.

Indentation applicators may be arranged in a paired parallel arrangement along the longitudinal axis of a carrier member, or may be arranged in a single row of indentation applicators along the longitudinal axis of a carrier member. Alternatively, a carrier member may take any shape and indentation applicators may be arranged in straight lines or in a random, or staggered arrangement. For example, a carrier member may be shaped as a planar rectangle or triangle, and the indentation applicators affixed to one side of the planar structure, with optionally a handle affixed to the opposite side of the planar structure. The indentation applicators may be affixed in any pattern desired to one side of the planar structure.

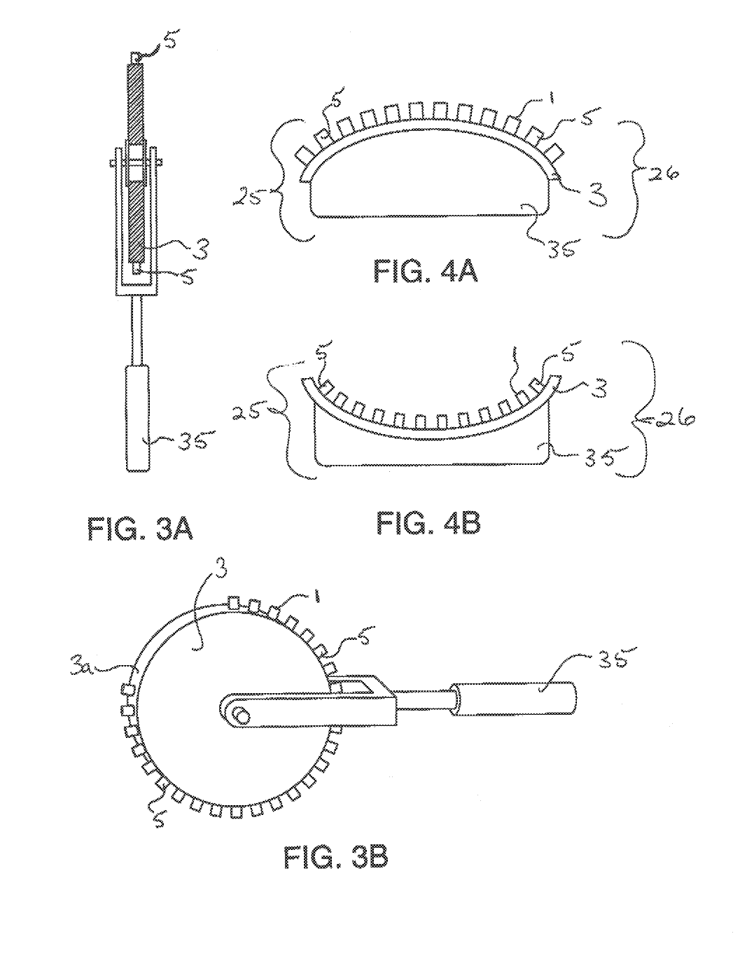

In an embodiment, shown in exemplary drawings at FIGS. 3A and 3B, the carrier member 3 of the delivery guide is a wheel, providing a rolling surface 3a to which one or more indentation applicators 5 are affixed. Connecting elements may or may not be present between one or more indentation applicators and the rolling surface of the wheel. One or more indentation applicators 5 extend perpendicularly and outward from the rolling surface 3a such that when the first end 1 of a first indentation applicator on the wheel contacts a surface, movement of the wheel along the surface causes the next in line indentation applicator on the rolling surface to contact the surface, followed by the third indentation applicator and so on. When used on a surface, the rolling wheel with indentation applicators provides a plurality of indentations, in a spaced apart relationship, to be formed in the contacted surface. The device having a rolling wheel carrier member may have a handle 35 attached through the center or axis of the wheel for moving the wheel across a surface.

In an embodiment, such as exemplified in FIGS. 4A and 4B, the injection guide comprises a curved carrier member 3 that may be any curved surface, whether convexly (FIG. 4A) or concavely (FIG. 4B) curved. For the convex shape embodiment, FIG. 4A, one or more indentation applicators 5 extend outwardly from the curved surface in a perpendicular or angled direction, depending on the carrier shape and/or the location of the indentation applicators 5. In use, the first end 1 of a first indentation applicator 5 on a first end 25 of the convex carrier 3, contacts a surface with sufficient force and for a sufficient time for an indentation in the surface to be formed, and with a rocking motion, the force is transferred in the direction toward second end 26 of the carrier 3 so that the next indention applicator in line is moved to contact the surface, and with force and time, a second indentation in formed in the surface, and then force is transferred so that the third indentation applicator 5 contacts the surface and forms the next indentation, and so of for as many indentations as are desired. In a similar manner, the device of 4B, with its concave carrier 3, may be fitted to a curved surface so that with the rocking motion described for the convex curved carrier of 4A, the individual indentation applicators may be moved so as to contact the surface and form indentations in the surface. For example, the concave carrier 3 is placed in contact with a convexly curved surface, and the first end 1 of a first indentation applicator 5 on a first end 25 of the concave carrier 3, contacts a surface with sufficient force and for a sufficient time for an indentation in the surface to be formed, and with a rocking motion, the force is transferred in the direction toward second end 26 of the carrier 3 so that the next indention applicator in line is moved to contact the surface, and with force and time, a second indentation in formed in the surface, and then the force is transferred so that the third indentation applicator 5 contacts the surface and forms the next indentation, and so of for as many indentations as are desired. Alternatively, two or more indentation applicators 5 on a concave carrier 5, or all of the indentation applicators 5 may be moved so that they contact the surface at one time with sufficient force and for a sufficient time to form a plurality of indentations in the surface. The curved carrier member with indentation applicators allows for a plurality of indentations, in a spaced apart relationship, to be formed in the contacted surface. The handle 35 may aid in the transfer of force from its source, such as from a medical practitioner's hand holding the handle, to the indentation applicators.

In the planar and curved carrier embodiments shown herein, a force is applied in a direction generally perpendicular to the surface, so that the indentation applicator first end 1 is forced against the surface to form an indentation in the surface. In the wheeled embodiment, the force is applied through the handle to the wheel in a sufficient amount so that the indentation applicator first end 1 is forced against the surface to form an indentation in the surface.

In an embodiment, a carrier member may comprise a contacting portion (not shown) that contacts the surface to leave a directional or numerical indication on the surface. For example, a portion of the carrier member may extend in a direction toward the surface and end at a distance from the carrier that corresponds to the length of the indentation applicator at the end of its first end 1. In use, when a sufficient force is applied for a sufficient length of time to form indentations by one or more indentation applicators, one or more contacting portions of a carrier member may leave an indentation indicating the beginning of the plurality of indentations and next closest or next in line indentation is made by a first or beginning indentation applicator. Alternatively, a contacting portion may have a marking solution applied so that when contacted to the surface, an inked design indicating a starting position for the first indentation applicator is applied to the surface. A contacting portion of the carrier member may be may be removably affixed to the carrier member or may be permanently present, such as molded with the carrier member. Alternatively, an indentation applicator may be shaped or marked to indicate that it is the first indentation applicator, or any other designation. In use, if for example, a row of indentations are formed and a different composition is injected in each indentation, having a marked starting indentation may be desirable for ease of recordkeeping and accurate understanding of what was injected along the row.

The first end 1 of an indentation applicator may have any desired shape. For example, the first end 1 may have an annulus or ring-shaped shape such that an annulus or ring-shaped indentation is formed in the contacted surface. The annulus end may be the only portion of the first end 1 that contacts the surface, or there may be portions of the first end 1, other than the annulus, that contacts the surface with the same or different force. For example, an annulus shape may be present in first end 1 and surrounding the annulus shape is another shape, for example, a star shape. The annulus shape may extend slightly further out, in a direction away from the second end 2, than the star shape, but not so far that the star shape will not contact the surface, but so that the star shape contacts the surface with less force than does the annular shape. In this way, if the star/annulus first end 1 contacts a marking solution, such as ink, and the ink is removed from the annulus shape and not the star shape portion, and the indentation applicator is then contacted to a surface, such as skin, with sufficient force for a sufficient length of time to form an indentation in the surface, the indentation formed comprises the annular shaped indentation surrounded by the design of a star formed by the ink impression, and a surface tattoo, is left on the surface. The present invention contemplates that washable and/or semi-permanent tattoos may be provided by indentation applicators having desired shapes, such as fanciful shapes. A portion of the indentation applicator first end 1 may provide an indentation in the surface that is used for reliable and uniform intradermal injections and a portion of the indentation applicator first end 1 may provide a number or design that is functions, as in providing an indicator of the indentation applicators in a schema of injections, or may provide distracting or entertaining tattoos for the recipient of the procedure. It is contemplated that such tattoos would distract or entertain children or adults undergoing a procedure. For example, a line of shaped indentation applicators may be used that provide a story line or that are numbered sequentially. When the health professional uses the line of indentations formed by the injection delivery guide to inject a series of allergens, for example with a different allergen at each indentation, the tattoo shapes may distract the receiving subject, or may serve as an indication of which allergen to inject at which indentation, and the allergen injected may be associated by the health professional with the tattoo shape on the skin surface.

In an embodiment, a marking or indication applicator may be used in place of indentation applicators, along with indention applicators or in a mixed plurality of indentation applicators and indication applicators place on a carrier. An indication applicator is similar in shape to an indentation applicator, and may be located location on a carrier member like an indentation applicator, and may function so that its first end (of an indication applicator) is not shaped to form an indentation, but instead, is shaped to leave a design or location indication and when coated with ink or a marking solution, will leave an indication on the contacted surface. For example, where both indention applicators and indication applicators are placed in a carrier, the indication applicators may be slightly shorter in length than the indentation applicators so that contacted with a surface and the force is applied to the carrier and the applicators, the indentation applicators form indentations and the indication applicators leave an ink mark on the surface where contacted. In use, an indication applicator is first contacted to ink or a marking solution, and then contacted to a surface, such as the skin of a human or animal, and when removed from the surface, only leaves the ink or marking solution on the surface, not an indentation.

An indication applicator may be of a shorter length than an indentation applicator so that when placed in a carrier member, the indication applicator contacts the surface with less force and does not form an indentation. For example, an indication applicator may be 0.1 to 30 mm shorter in length than an indentation applicator so that when applied to a surface, the sufficient force to create indentations on the surface with an indentation applicator causes an indication applicator to leave a mark or indication on the surface contacted. An indication applicator may be self-inking or may be contacted with a marking solution prior to use in contacting a surface. In an embodiment, the connecting element may be shorter in length, so that in use, one or more longer connecting elements are affixed to the indentation applicators and shorter connecting elements are affixed to indication applicators, so that when a sufficient force is applied for a sufficient time, the indentation applicators create indentations in the surface, and the indication applicators creates indications or markings only on the surface.

One or more indication applicators may be affixed in a carrier member with one or a plurality of indentation applicators. For example, with removable applicators, applicators having different functions, such as marking or forming indentations, may be used interchangeably. For example, a carrier member may have sevent (7) receiving sites formed on one side, such as the side that faces the surface when contacting a surface, for example, see FIG. 2D, 3a. The receiving sites may be numbered 1 to 7, and are in a straight line down the longitudinal axis of a bar-shaped carrier member. At site 1 an indication applicator may be affixed that has a first end shaped as a number 1, so that when that first end 1, having a coating of marking solution, contacts the surface and is removed, the image of the number 1 is then present on the surface. At sites 2-7, indention applicators are affixed. When a sufficient force for a sufficient time is applied to a carrier member and its applicators, the indentation applicators form indentations in surface that are visible when the indentation applicators are removed from contacting the surface. For example, in another use, at sites 3 and 5, no applicator is affixed, so that the receiving sites at 3 and 5 are empty, and after contacting with such an arranged injection delivery guide device, the surface shows a row with a number 1 followed by two indentations, a blank area, an indentation, a blank area, followed by five indentations spaced regularly apart. In an embodiment, such patterns of indentation applicators and indication applicators may be molded into a unitary carrier member, so that that particular molded injection delivery guide device always provides the same pattern of indications and/or indentations to the surface.

In an aspect of the invention, indentation applicators and/or indication applicators may be spaced apart from each other when affixed to a carrier member. For example, indentation applicators and/or indication applicators may be spaced apart from each other by about from 0.2 cm to 5 cm, from about 0.2 cm to 10 cm, and any amount thereinbetween. Such spacing may be altered as desired, and may be changed by removably affixing the applicators in any pattern or in no particular pattern to a carrier member.

The shape, size and depth of the indentation formed by an indentation applicator is dependent on the indentation applicator. For example, the shape and size of the first end 1 of the indentation applicator may determine the shape and size of the indentation, and depth of the indentation may be dependent on the length of the indentation applicator, in that a particular indentation applicator may be longer than other indentation applicators affixed to the carrier member, or the first end of indentation applicator may protrude to a position that when the surface is contacted, a deeper indentation is formed. The depth of the indentation may be affected by the applied sufficient force used to contact the indentation applicator with the surface, or it may be affected by the surface which is contacted. In general use, it is contemplated that the indentation applicators would contact the surface with a uniform force so that all the indentations formed would be of a constant and even depth, size and shape. For example, annular (ring-shaped) indentations may be made in a surface, such as skin of a human or animal. The raised central section skin, within the annulus, is of a determined size. For example, the diameter of the annulus may be from about 1 mm to 3 cm, from about 1 cm to about 3 cm, from about 2 cm to about 3 cm, from about 3 mm to about 3 cm, from about 1 mm to about 1 cm, from about 1 mm to about 75 mm, from about 1 mm to about 50 mm, from about 1 mm to about 25 mm, from about 1 mm to about 15 mm, from about 1 mm to about 10 mm, from about 1 mm to about 7 mm, from about 1 mm to about 7 mm, from about 1 mm to about 6 mm, from about 1 mm to about 5 mm, from about 1 mm to about 4 mm, from about 1 mm to about 3 mm, from about 1 mm to about 2 mm, from about 2 mm to about 7 mm, from about 2 mm to about 6 mm, from about 12 mm to about 5 mm, from about 2 mm to about 4 mm, from about 2 mm to about 3 mm, and ranges thereinbetween.

A kit of the present invention may comprise an injection delivery guide device and a tray to hold the injection delivery guide device comprising at least one carrier member and one or more indentation applicators and/or one or more indication applicators, and optionally a handle for at least one carrier member. The tray may have a lid, such that when joined or the lid is closed, the tray and lid form a closed container in which the injection delivery guide device can be contained. In an embodiment, the tray may sit within a box with or without a lid, or within a larger bottom tray that mates with a lid to form a closed container for the tray and injection delivery guide device. Such kits may be useful in transporting the injection delivery guide device or for autoclaving, sterilizing or cleaning the injection delivery guide device. A kit may further comprise one or more vials or containers of the solution(s) to be administered after indentations are formed, and/or may contain marking solutions to provide indications along with the indentations. A tray may contain wells that can match or mate with the first end 1 of indentation applicators and/or indication applicators, such that the wells may be filled with marking solutions or cleansing solutions,such as alcohol. When the first end(s) 1 are contacted with the solutions in the wells, the first end(s) 1 may be coated with marking solution(s) or may be cleansed.

Injection delivery guide devices, trays and components may be made of any material such as plastics, metals, aluminum, and may be provided as one-use disposable devices or may be reusable. An injection delivery guide device may be made from one or more different materials, or individual components, such as the handle or indentation applicator, or sections of an indentation applicator may be made from different materials. For example, a hard rubber coating may be placed on the first end of a plastic indentation applicator, or a metal screw section may be present on the second end of a plastic indentation applicator. Injection delivery guide devices or systems may be sterile when provided, or may be cleaned by known methods for devices for dermal and topical use. The devices may be molded or cast so as to be a one piece injection delivery guide device comprising a carrier member with or without indicator elements, one or more indentation applicators, optionally one or more connecting elements, one or more indication applicators, and/or one or more handles. Alternatively, the injection delivery guide device may be assembled from individual parts comprising a carrier member with or without indicator elements, one or more indentation applicators, optionally one or more connecting elements, one or more indication applicators, and/or one or more handles.

Injection delivery guide devices of the present invention may be used in any procedure where controlled injection is desired. For example, if an opening is provided through the central portion of an indentation applicator or an indication applicator, the applicators may serve as injection guides for a needle. A use of the injection delivery guide device is to provide uniform, regularly spaced indentations in a surface, such as the skin of a human or animal, for controlled intradermal injection of solutions comprising agents.

Intradermal skin testing for allergen reactions comprises quantitative and qualitative measurements. For the test to be accurate, a predetermined amount of an allergen must be injected within the intradermal space of the skin in a defined shape. The test relies on the response of the recipient to alter the original site of injection, thus if the original site of injection varies or the amount injected varies, when the site is inspected at some time period after injection, such as minutes, hours, days or weeks, it is unclear what caused the change at the site of injection. Accurate interpretation of the response of the recipient at the injection site is dependent on the proficiency of the healthcare provider who performed the intradermal injection. An injection delivery guide device provides an injection site that is reliably uniform in depth, shape and location.

For example, in use, an injection delivery guide device forms a row of annular indentations, each with a defined shape and size central skin section, into which a controlled intradermal injection is made. The shape and size of the central skin section provides a defined space and size for guiding the placement of the injection, the amount of solution to be injected and controls the shape of the bolus formed by the injection. The annular indentation is only temporary, and once the skin resumes its normal height and the indentation is resolved, all of the injection sites will be seen as raised, uniformly sized, regularly spaced bolus injection sites. The sites may have been marked by the injection delivery guide device with a marking solution for ease of location of the first or all of the sites.

It is very difficult for a healthcare professional to provide uniform administration of the same amount of solution to several injection sites, particularly when the volume of solution injected is too small to accurately measure in a syringe. Additionally, it is difficult to form a uniformly shaped bolus of injected solution. Too much solution may cause a reaction by the recipient that looks like a more vigorous response to the solution than would be seen had the correct amount of solution been administered. A recipient may respond to an injected allergen with a wheal and flair response, and it is important to measure the size and shape of the wheal and flair made in the response. If the initial bolus of solution was not a controlled shape with defined sharp demarcated edges, that site may be later interpreted to be a wheal and flair response, when in reality, there was no or little response and the measurement is only an artifact of the test procedure and not a response by the recipient. For accurate testing and quality assurance standards to be met, it is important than accurate and uniform intradermal administration techniques are followed.

For example, in allergen testing, the initial intradermal bolus injection size and shape, which forms the initial test wheal, is critical for an accurate test. The test is valid only if the initial test wheals formed by the injected solution bolus is a uniform size, for example 4 mm in diameter, with a sharp demarcated edge. The initial intradermal test wheal diameter is directly related to the volume of solution injected, and that volume is related to the resulting diameter of the reaction by the recipient. For example, to create a 4 mm initial test wheal with a sharp demarcated edge, 0.01 to 0.02 mL of solution needs to be injected intradermally into the skin. This 0.01 to 0.02 mL cannot be accurately administered regularly using an unguided technique. The syringe operator cannot determine, just by looking at the syringe markings that such a small amount was delivered at every injection site. The amount is too small to be measured by markings on a syringe. If the exact 0.01 to 0.02 mL of solution is drawn into the syringe, some of the volume is lost due to adherence to the needle, syringe, possible leakage of the solution as it is injected, resulting in less being injected and a smaller than desired wheal.

Proficiency in creating uniform, consistent intradermal test wheals is especially critical when treatment solutions are formulated based on the exact measurement of the resulting wheal created by the response of the recipient. The otolaryngic allergy specialty profession uses the measurement of the resulting wheal as the basis for treatment solution calculations. The solutions are formulated based on the endpoint dilution, which is determined by the measurement of the intradermal resulting wheal. The validity of the endpoint result is dependent on creating an initial test wheal that is no smaller and no larger than a predetermined diameter, such as 4 mm. If more or less volume of the allergen dilution was intradermally injected, misinterpretation of the resulting wheal may result in assignment of an incorrect endpoint to the allergen being tested. This incorrect test result and misinterpretation of the endpoint dilution could result in the recipient, a patient, receiving treatments with solutions that are too strong or too weak. The administration variables should not be the cause of inadequate or dangerous procedures performed on humans or animals.

Before the present invention, there was no method or device to provide reliable intradermal injections, such as in allergen testing. Healthcare providers could not make uniform test wheals of injected solutions reliably. It takes about 0.01 to 0.02 mL of solution to make a 4 mm test wheal. Even well-trained and experienced healthcare providers were forced to approximate the test wheals. The approximations resulted in creation of wheals that were not uniform and not reproducible. A healthcare provider may be injecting from about 10 to 30 different solutions, using different vials and syringes for each solution and injection. Uniformity is very hard to achieve under such variety of movements. Each intradermal test wheal, even when applied at the same time, may be different from the others applied in the same timeframe. Using different healthcare providers in the same procedure only increases the variability of the test. This variability is of concern, because the final calculations are based on an assumption that all solutions were delivered in a reliably uniform fashion and the differences seen between wheals is due to the response by the recipient. For example, if the diameter of the negative control wheal is not the same as an initial test wheal, the resulting wheal cannot be compared accurately to the negative control wheal and the skin test cannot be validated. Every initial wheal must be uniform and provide the same amount of solution as the initial control wheals, positive and negative, or the accuracy and reliability of the test cannot be assured. With the use of the devices of the present invention, such accuracy and reliability can be met.

An example of use of an injection delivery guide device of the present invention in the testing of allergen response is provided. The test site is first cleaned with alcohol and allowed to dry. An injection delivery guide device comprising at least a carrier member and at least one indentation applicator is applied perpendicularly to the skin, so that the first end of the indentation applicator contacted the skin. A force sufficient to create an indentation in the skin by the indentation applicator is applied for a sufficient amount of time to create an indentation in the skin by the indentation applicator, the force is provided, for example, by a healthcare provider. When the injection delivery guide device is removed from the skin after a sufficient time of application, which may be from 1 to 10 minutes, a pattern of indentations is seen on the skin, the pattern being determined by the placement of the one or more indentation applicators in the carrier member. For example, there may be from 1 to 30 uniform, 4 mm to 6 mm diameter annular rings indented in the skin, each spaced from 0.4 to 4 cm apart.

The indentation formed is temporary and may remain visible for 5 to 20 minutes. For example, the indentation is an annular ring with an outer diameter of 6 mm and an inner diameter of 4 mm, such as that a raised 4 mm inner area of skin is visible to the eye.

A small amount of a particular allergen solution is drawn into a syringe, for example, about 0.04 to 0.05 mL, though less than this amount will be injected in any one location. An injected amount is from about 0.01 to 0.02 mL. The recipient patient's arm is grasped from behind and the skin pulled tightly. The syringe needle is placed at the edge of the visible indentation, on the inner side of the annulus, next to the raised inner 4 mm area, keeping the syringe parallel to the indentation. The bevel of the needle is inserted down into the superficial intradermal layer of skin. Gentle pressure is applied to the plunger of the syringe as the solution leaves the needle and fills the test wheal defined by the raised inner 4 mm section. The test wheal, containing the injection solution bolus, is a defined 4 mm diameter wheal with sharp edges. It is recommended to make at least a second test wheal at a known location, for duplicate or repeated test sites to ensure greater accuracy in the resulting measurements.

A new syringe containing a different solution is provided and the injections are repeated at the other indentations until all solutions are administered and a patterned test area is created on the skin of the recipient. The test wheals are spaced apart clearly and regularly, and the test wheals are uniform in diameter. In about 10-20 minutes, the recipient's response is measured and recorded. The test results are reliable, consistent and as reproducible as are possible with immune testing. The interpretation of the test and the quality assurance standards are met by removing most of the test procedure variable from the procedure by use of an injection delivery guide device.

As used herein, an indentation may comprise any shape. An indentation may comprise an indented area of surface, such as skin, that surrounds and defines a non-indented area of surface or skin. An indentation may comprise only an indented area of surface, such as skin. How the indentation is to be used may determine the whether the indented surface area surrounds, encloses or defines a non-indented area of surface, wherein the defined non-indented area is the focus of the use, or whether the indented area only is the focus of the use. An injection amount may fill an area of indentation or it may be of a size that the bolus area is smaller or larger that the area of the indentation.

The measurements given in this example are for illustration only and are not to be considered as limiting to the invention. One skilled in the art can understand that the indentations may be made with any desired size or shape, and the ones described herein are for clarity, not limitation.

Though the disclosure of uses of the present invention has considered allergen testing, the invention is not to be limited to this embodiment only, and other uses, such as those where the route of administration is intradermal, are contemplated by the present invention. For example, it has been found that the delivery of some vaccines or medicines is more efficacious if delivered intradermally and not by subcutaneous or intramuscular injection administration routes. The volume of vaccine or medication is reduced, leading to dose-sparing. These uses would also be aided by use of an injection delivery guide device of the present invention to provide controlled intradermal delivery in a consistent and reliable manner.

It must be noted that, as used in this specification and the appended claims, the singular forms “a,” “an,” and “the” include plural referents unless the context clearly dictates otherwise.

All patents, patent applications and references included herein are specifically incorporated by reference in their entireties.

It should be understood, of course, that the foregoing relates only to preferred embodiments of the present invention and that numerous modifications or alterations may be made therein without departing from the spirit and the scope of the invention as set forth in this disclosure.

Examples in the present invention are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

What is claimed is:

1. An injection delivery guide, comprising a carrier having affixed at least one indentation applicator having a first end for contacting skin, wherein the first end is blunt and does not puncture or cut skin.

2. The guide of claim 1, wherein an indentation applicator is affixed to the carrier by a connecting element located between a second end of the indentation applicator and the carrier.

3. The guide of claim 1, wherein the first end has a diameter of from about 1 mm to about 8 mm.

4. The guide of claim 1, wherein there are from 2 to 30 indention applicators.

5. The guide of claim 1, wherein the indention applicators are affixed in a straight line along a longitudinal axis of the carrier.

6. The guide of claim 1, wherein the indention applicators are affixed in two or more straight lines parallel to a longitudinal axis of the carrier.

7. The guide of claim 1, wherein one or more indentation applicators are affixed to a wheel carrier.

8. The guide of claim 1, wherein one or more indentation applicators are affixed to a curved carrier.

9. The guide claim 6, wherein the curved carrier has a convex shape.

10. The guide of claim 6, wherein the curved carrier has a concave shape.

11. The guide of claim 1, wherein the indention applicators are spaced apart from each other approximately between 0.2 cm to 5 cm.

12. The guide of claim 1, further comprising a handle attached to the carrier.

13. The guide of claim 1, wherein at least one indentation applicator is optionally removable from the carrier.

14. The guide of claim 1, further comprising at least one indication applicator.

15. A kit, comprising an injection guide of claim 1 and a tray.

16. The kit of claim 15, further comprising solutions to be injected, marking solutions, vials for holding solutions and optionally, instructions for use of the guide.

17. A method of using an injection delivery guide, comprising, applying, with force, to a surface for a sufficient time period, an injection delivery guide comprising at least one indentation applicator having a first end for contacting skin, wherein at least one indentation applicator contacts the surface and forms an indentation in the surface.

18. The method of claim 17, wherein the surface is skin of a human or animal.

19. The method of claim 18, further comprising,
a) after forming at least one indentation in the surface that defines an area in the surface, removing the injection delivery guide from the surface;
b) inserting a needle of a syringe containing a liquid at the exterior edge of the indentation into the superficial intradermal layer of the skin; and
c) injecting the liquid into the intradermal layer such that the liquid fills the area in surface defined by the indentation.

20. The method of claim 19, wherein the liquid is a solution comprising an allergen, an immunogen, a vaccine, medication, therapeutic agent, or a peptide.
